SEQUENCE LISTING (7 sequences)

SEQ ID NO: 1 - protein, 349 aa, Homo sapiens
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 2 - protein, 17 aa, synthetic construct
  source 1..17: note = The Thumb domain of WNT7B
KCHGVSGSCTTKTCWTT

SEQ ID NO: 3 - protein, 14 aa, synthetic construct
  source 1..14: note = The index domain of WNT7B
QCNCKFHWCCFVKC

SEQ ID NO: 4 - protein, 31 aa, synthetic construct
  source 1..31: note = synthetic peptide used for promoting bone formation
KCHGVSGSCTTKTCWTTQCNCKFHWCCFVKC

SEQ ID NO: 5 - protein, 28 aa, synthetic construct
  source 1..28: note = synthetic peptide used for promoting bone formation
KCHGVSGSCGPGGDQCNCKFHWCCFVKC

SEQ ID NO: 6 - protein, 19 aa, synthetic construct
  source 1..19: note = synthetic peptide used for promoting bone formation
GPGGDQCNCKFHWCCFVKC

SEQ ID NO: 7 - protein, 14 aa, synthetic construct
  source 1..14: note = synthetic peptide used for promoting bone formation
GPGGDKCHGVSGSC

(19) United States
(12) Patent Application Publication
(10) Pub. No.: US 2026/0035419 A1
(43) Pub. Date: Feb. 5, 2026

(54) BONE FORMATION PROMOTING POLYPEPTIDE AND APPLICATION THEREOF

(71) Applicant: Sichuan University, Chengdu (CN)

(72) Inventors: Fanyuan YU, Chengdu (CN); Feifei LI, Chengdu (CN); Peng YU, Chengdu (CN); Chuyi HAN, Chengdu (CN); Yitian WANG, Chengdu (CN); Xue YANG, Chengdu (CN); Ling YE, Chengdu (CN)

(21) Appl. No.: 18/949,459

(22) Filed: Nov. 15, 2024

(30) Foreign Application Priority Data
Jul. 30, 2024 (CN) 202411025205.5

(51) Int. Cl. C07K 14/47; A61K 38/00; A61K 45/06; A61P 19/10

(52) U.S. Cl. CPC C07K 14/4702; A61K 45/06; A61P 19/10; A61K 38/00; C07K 2319/01; C07K 2319/20

(57) ABSTRACT

The present invention belongs to the technical field of biopharmaceutical materials, and specifically relates to a bone formation promoting polypeptide and application thereof. Specifically, the present invention provides a polypeptide or the variant thereof for promoting bone formation, bone regeneration, and bone repair, and/or preventing and/or treating osteoporosis, a fusion protein and a pharmaceutical composition comprising such a polypeptide or the variant thereof, and pharmaceutical use of such a polypeptide or the variant thereof and fusion protein.

### INCORPORATION OF SEQUENCE LISTING

This application contains a sequence listing submitted in Computer Readable Form (CRF). The CRF file contains the sequence listing entitled “PA7420007-SequenceListing.xml”, which was created on Nov. 12, 2024, and is 7,592 bytes in size. The information in the sequence listing is incorporated herein by reference in its entirety.

### TECHNICAL FIELD

The present invention belongs to the technical field of biopharmaceutical materials, and specifically relates to a bone formation promoting polypeptide and application thereof.

### BACKGROUND

At present, the only available bone formation promoting drug in clinical practices is parathyroid hormone derived peptide (PTH 1-34, Teriparatide). Evidence-based clinical data show that intermittent use of Teriparatide is an effective method for treating osteoporosis. However, its benefits for fracture healing and bone regeneration still need to be evaluated in more clinical studies (Eben G. E., Clifford J. R. Emerging insights into the comparative effectiveness of anabolic therapies for osteoporosis. Nat. Rev. Endocrinol. 17, 31-46 (2021)).

Previous studies have shown that the classical WNT/β-catenin signaling pathway plays a wide role in bone homeostasis, bone development, bone regeneration, and bone metabolism. Activating the classical WNT pathway can promote bone formation in animal models (Tristan W. F. et al, Development of selective bispecific Wnt mimetics for bone loss and repair. Nat. Commun. 12, 3247 (2021)). Therefore, the classical WNT signaling pathway is also an important target for the development of bone regeneration drugs, and the classical WNT agonists such as romosozumab and recombinant WNT3A (rhWNT3A), etc., have been used in the field of bone formation promotion. However, in the process of practical applications and clinical transformations, it has been found that the use of such classical WNT agonist drugs may lead to risk of tumorigenesis and severe adverse reactions due to excessive activation of the classical WNT/β-Catenin signaling pathway. These non-ignorable risks limit the development and practical applications of related drugs.

Therefore, there is an unmet need in this field for the development of new therapeutic regimens and drugs that play the bone formation promotion role without activating the classical WNT signaling pathway.

### SUMMARY

Previous studies of the inventors have shown that the bone anabolic action of WNT7B (Wnt family member 7B) is independent of the presence of β-Catenin and there is no risk of activating the classical WNT pathway, (Fanyuan Y. et al, Wnt7b-induced Sox11 functions enhance self-renewal and osteogenic commitment of bone marrow mesenchymal stem cells. Stem Cells. 38, 1020-1033 (2020)). Therefore, the present invention intends to demonstrate that the WNT7B-derived polypeptides can play the bone formation promotion role in models of osteoporosis and critical bone defects without activating the classical WNT signaling pathway, through the development of polypeptides derived from the Thumb and index domains of WNT7B, and the therapeutic effect of such derived polypeptides on osteoporosis and the efficacy of promoting fracture repair and regeneration have been verified in multiple species such as mice and pigs, etc. This effect is closely related to the self-renewal and osteogenic differentiation enhancement of MSCs, and is mediated by the activation of the Reck/Gpr124/Ca²⁺/Nfatc1 signaling axis by WNT7B-derived polypeptides. Therefore, the present invention intends to address the drawbacks of current WNT activators in clinical applications and provide novel bone formation promoting drugs, filling the gaps in the development of low-risk bone formation drugs and providing new strategies for the latest generation of treatment of bone loss and bone injury diseases such as osteoporosis and fracture healing, etc.

### Isolated Polypeptide or the Variant Thereof

In one aspect, the present invention provides an isolated polypeptide or a variant thereof, wherein the polypeptide consists of amino acids at positions 200-217 and amino acids at positions 323-336 of WNT7B protein;

the variant differs from the polypeptide from which the variant is derived in substitutions (e.g. conservative substitutions), deletions, or additions of one or more (including but not limited to, 1, 2, 3, 4, 5 or more) amino acid residues, and retains biological function of the polypeptide from which the variant is derived.

In this text, the biological function of the polypeptide or the variant thereof of the present invention includes but is not limited to promoting bone formation, bone regeneration, bone repair, and/or treating osteoporosis.

In some embodiments, the WNT7B protein is of human origin. In some embodiments, the WNT7B protein has an amino acid sequence as shown in SEQ ID NO:1.

In some embodiments, the isolated polypeptide of the present invention is composed by directly linking amino acids at positions 200-217 to amino acids at positions 323-336 of WNT7B protein.

In some embodiments, the amino acids at positions 200-217 of WNT7B protein correspond to the Thumb domain of WNT7B, and its sequence is as shown in SEQ ID NO:2.

In some embodiments, the amino acids at positions 323-336 of WNT7B protein correspond to the index domain of WNT7B, and its sequence is as shown in SEQ ID NO:3.

In some embodiments, the isolated polypeptide comprises an amino acid sequence as shown in SEQ ID NO:4.

In some embodiments, the variant comprises an amino acid sequence as shown in SEQ ID NOs: 5-7.

### Fusion Protein

In another aspect, the present invention provides a fusion protein comprising the isolated polypeptide (or the variant thereof) of the present invention and an additional polypeptide.

In some embodiments, the additional polypeptide is selected from a protein tag, a targeting moiety, or any combinations thereof.

In this text, the protein tag is well-known in the art, and examples of which include but are not limited to His, Flag, GST, MBP, HA, Myc, GFP, or biotin, and those skilled in the art know how to select appropriate protein tags according to desired purposes such as purification, detection, or tracing.

In this text, the term “a targeting moiety” refer to a domain that can guide the polypeptide (or the variant thereof) of the present invention to a desired location, which can be a specific tissue, a specific cell, or even a specific intracellular location (such as nucleus, ribosome, endoplasmic reticulum, lysosome, or peroxysome). Those skilled in the art know how to design corresponding targeting domains based on the characteristics of the desired locations. In some embodiments, the targeting moiety includes a ligand, a receptor, an antibody, or a binding domain thereof.

In some embodiments, the additional polypeptide is linked to the N-terminus or C-terminus of the polypeptide (or the variant thereof) of the present invention optionally via a linker. In some embodiments, the linker is a sequence containing one or more (e.g. 1, 2, 3, 4, or 5) amino acids (e.g. Gly or Ser).

### Preparation of a Polypeptide and a Fusion Protein

The polypeptide or the variant thereof or the fusion protein of the present invention is not limited by its production method. For example, it can be produced by a genetic engineering method (a recombinant technology) or by a chemical synthesis method, such as produced by solid-phase synthesis on a suitable resin.

In another aspect, the present invention provides an isolated nucleic acid molecule comprising a nucleotide sequence encoding the polypeptide or the variant thereof or the fusion protein of the present invention.

In another aspect, the present invention also provides a vector comprising the isolated nucleic acid molecule as described above. The vector of the present invention can be a cloning vector or an expression vector. In some embodiments, the vector of the present invention is, for example, plasmids, cosmids, phages, and cosmids, etc.

In another aspect, the present invention also provides a host cell comprising the isolated nucleic acid molecule or the vector of the present invention. Such a host cell includes but is not limited to prokaryocytes such as E. coli cells, and eukaryocytes such as yeast cells, insect cells (e.g. Sf9 cells), plant cells and animal cells (e.g. mammalian cells, such as mouse cells, and human cells, etc.).

In another aspect, the present invention also provides a method for preparing the polypeptide or the variant thereof or the fusion protein of the present invention, comprising culturing the host cell of the present invention under conditions that allow expression of the polypeptide or the variant thereof or the fusion protein, and recovering the polypeptide or the variant thereof or the fusion protein from the cultured host cell culture.

### Pharmaceutical Composition

In another aspect, the present invention provides a pharmaceutical composition comprising the isolated polypeptide (or the variant thereof), the fusion protein, the isolated nucleic acid molecule, the vector or the host cell of the present invention, and a pharmaceutically acceptable carrier and/or an excipient.

In some embodiments, the pharmaceutical composition comprises one or more of the isolated polypeptide (or the variant thereof) or the fusion protein of the present invention.

The polypeptide (or the variant thereof), the fusion protein or the pharmaceutical composition of the present invention can be formulated into any dosage forms known in the medical field, such as tablets, pills, suspensions, emulsions, solutions, gels, capsules, powders, granules, elixirs, lozenges, suppositories, injections (including injection solutions, lyophilized powders), etc. In some embodiments, the polypeptide (or the variant thereof), fusion protein, or pharmaceutical composition of the present invention can be formulated into injection solutions or lyophilized powders.

In addition, the polypeptide (or the variant thereof) or the fusion protein of the present invention may be present in the pharmaceutical composition in the form of unit doses for ease of administration.

The polypeptide (or the variant thereof), the fusion protein, or the pharmaceutical composition of the present invention can be administered by any suitable methods known in the art, including but not limited to oral, buccal, sublingual, intraocular, local, parenteral, rectal, intravaginal, cisternal, inguinal, intravesical, topical (e.g. powder, ointment, or droplet), or nasal routes. However, for many therapeutic uses, the preferred administration route/method is parenteral administration (e.g. intravenous injection, subcutaneous injection, intraperitoneal injection, and intramuscular injection). Those skilled in the art should understand that the administration route and/or method will be varied depending on the intended purposes. In a preferred embodiment, the polypeptide (or the variant thereof), fusion protein, or pharmaceutical composition of the present invention is administered via intravenous infusion or injection.

The polypeptide (or the variant thereof), the fusion protein, or the pharmaceutical composition provided by the present invention can be used alone or in combination, or in combination with an additional pharmaceutically active agent (e.g. a bone formation promoting drug). Such an additional pharmaceutically active agent can be administered before, concurrently with, or after the administration of the polypeptide (or the variant thereof), fusion protein, or pharmaceutical composition of the present invention.

In some embodiments, the pharmaceutical composition optionally further comprises an additional pharmaceutically active agent, such as an other drug that has effects of promoting bone formation, bone regeneration, and/or bone repair, such as bone formation promoting drugs, such as Teriparatide.

### Therapeutic Use

The inventors of this application have discovered for the first time that by assembling the Thumb and index domains of WNT7B, the obtained WNT7B-derived polypeptides can play the bone formation promotion role in models of osteoporosis and critical bone defects without activating the classical WNT signaling pathway.

Thus, in another aspect, the present invention also relates to use of the isolated polypeptide (or the variant thereof), the fusion protein, the isolated nucleic acid molecule, the vector or the host cell of the present invention in preparation of a drug, wherein the drug is configured to promote bone formation, bone regeneration, and bone repair, and/or prevent and/or treat osteoporosis in a subject.

In some embodiments, the subject can be mammals, such as humans or mice. In some embodiments, the subject is any subject requiring to promote bone formation, bone regeneration, and/or bone repair. In some embodiments, the subject has or is suspected to have osteoporosis, or is at risk of developing the aforementioned diseases.

### Definitions of Terms

In the present invention, unless otherwise specified, the scientific and technical terms as used herein have the meanings commonly understood by those skilled in the art. Moreover, the laboratory procedures of virology, biochemistry, and immunology as used herein are all routine procedures widely used in their respective fields. Meanwhile, in order to better understand the present invention, definitions and explanations of relevant terms are provided below.

As used herein, WNT7B is a member of the WNT gene family, which consists of structurally related genes encoding the secretory signaling proteins. These proteins are associated with tumorigenesis and various development processes, including the regulation of cell fates and patterns during embryogenesis. WNT7B is well-known to those skilled in the art, and its sequence can be found in various public databases, such as NCBI GENBANK database accession number: NM 058238.3.

As used herein, when referring to the amino acid sequence of WNT7B protein, the sequence as shown in SEQ ID NO: 1 is used for description. For example, the expression “amino acid residues at positions 200-217 of WNT7B protein” refers to the amino acid residues at positions 200-217 of the polypeptide as shown in SEQ ID NO: 1. However, those skilled in the art can understand that mutations or variations can be naturally generated or artificially introduced in an amino acid sequence of WNT7B without affecting its biological functions. Therefore, in the present invention, the term “WNT7B” and similar expressions thereof should encompass all such sequences, including for example sequences as shown in SEQ ID NO:1, and natural or artificial variants thereof. Moreover, when describing the sequence fragments of WNT7B protein, it includes not only the sequence fragments of SEQ ID NO: 1, but also the corresponding sequence fragments in the natural or artificial variants thereof. For example, the expression “amino acid residues at positions 200-217 of WNT7B protein” includes the amino acid residues at positions 200-217 of SEQ ID NO:1, as well as the corresponding fragments in the natural or artificial variants thereof. According to the present invention, the expression “corresponding sequence fragments” or “corresponding fragments” refers to the fragments located at the equivalent positions in the compared sequences when the sequences are subjected to optimal alignment, that is, when the sequences are aligned to obtain the highest percentage of identity.

As used herein, the term “isolated” or “be isolated” refers to one is obtained through artificial means from its natural state. If a certain “isolated” substance or component appears in nature, it may be due to a change in natural environment where it exists, or the substance is isolated from the natural environment, or both. For example, a certain non-isolated polynucleotide or polypeptide naturally exists in a living animal, while the high-purity identical polynucleotide or polypeptide isolated from this natural state is thus called “isolated”. The term “isolated” or “be isolated” does not exclude the presence of artificial or synthetic substances therein, nor does it exclude the presence of other impure substances that do not affect the activity of the substance.

As used herein, the term “vector” refers to a nucleic acid delivery vehicle that can insert the polynucleotides therein. When a vector enables the expression of the protein encoded by the inserted polynucleotide, the vector is called as an expression vector. A vector can be introduced into host cells through transformation, transduction, or transfection, allowing the genetic material elements carried by them to be expressed in the host cells. The vector is well-known to those skilled in the art, including but not limited to plasmids; phagemids; cosmids; artificial chromosomes, such as yeast artificial chromosomes (YACs), bacterial artificial chromosomes ((BACs), or P1-derived artificial chromosomes (PACs); bacteriophages such as lambda bacteriophage or M13 bacteriophage, as well as animal viruses, etc. Animal viruses that can be used as the vector include but are not limited to retroviruses (including lentiviruses), adenoviruses, adeno-associated viruses, herpesviruses (e.g. herpes simplex virus), poxviruses, baculoviruses, papillomavirus, and papovaviruses (e.g. SV40). A vector can contain multiple elements that control expression, including but not limited to promoter sequences, transcription initiation sequences, enhancer sequences, selection elements, and reporter genes. In addition, the vector may also contain origin of replication.

As used herein, the term “host cells” refers to cells that can be used to introduce a vector, including but not limited to prokaryocytes such as Escherichia coli or Bacillus subtilis, etc.; fungal cells such as yeast cells or Aspergillus, etc.; insect cells such as S2 Drosophila cells or Sf9, etc.; or animal cells such as fibroblasts, CHO cells, COS cells, NSO cells, HeLa cells, BHK cells, HEK 293 cells, or human cells, etc.

As used herein, the term “conservative substitutions” means the amino acid substitutions that do not adversely affect or alter the expected properties of proteins/polypeptides containing an amino acid sequence. For example, conservative substitutions can be introduced through standard techniques known in the art, such as site-directed mutagenesis and PCR mediated mutagenesis. Conservative amino acid substitutions include the substitutions of replacing amino acid residues with amino acid residues that have similar side chains, such as the substitution using residues that are physically or functionally similar to the corresponding amino acid residues (e.g. having similar sizes, shapes, charges, chemical properties, including the ability to form covalent or hydrogen bonds, etc.). Families of amino acid residues with similar side chains have been defined in the art. These families include amino acids with alkaline side chains (e.g. lysine, arginine, and histidine), acidic side chains (e.g. aspartic acid, and glutamic acid), uncharged polar side chains (e.g. glycine, asparagine, glutamine, serine, threonine, tyrosine, cysteine, and tryptophan), non-polar side chains (e.g. alanine, valine, leucine, isoleucine, proline, phenylalanine, methionine), beta branched side chains (e.g. threonine, valine, isoleucine), and aromatic side chains (e.g. tyrosine, phenylalanine, tryptophan, and histidine). Therefore, it is preferable to replace the corresponding amino acid residue with another amino acid residue from the same side chain family. The methods for identifying conservative substitutions of amino acids are well-known in the art (see, for example, Brummell et al., Biochem. 32:1180-1187 (1993); Kobayashi et al, Protein Eng. 12 (10): 879-884 (1999); and Burks et al, Proc. Natl Acad. Set USA 94:412-417 (1997), which are incorporated herein by reference).

The writing of the twenty conventional amino acids involved herein follows conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, the terms “polypeptide” and “protein” have the same meaning and can be used interchangeably. Moreover, in the present invention, the amino acids are usually represented by single letter and three-letter abbreviations well-known in the art. For example, alanine can be represented by A or Ala.

As used herein, the term “a subject” includes but is not limited to various animals, particularly mammals such as humans. In some embodiments, the subject (e.g. humans) suffers from osteoporosis. As used herein, the term “a pharmaceutically acceptable carrier and/or an excipient” refers to a carrier and/or an excipient that is pharmacologically and/or physiologically compatible with the subjects and active ingredients, which is well-known in the art (see, for example, Remington's Pharmaceutical Sciences. Edited by Gennaro AR, 19^{th} ed. Pennsylvania: Mack Publishing Company, 1995), and includes but is not limited to: pH regulators, surfactants, ionic strength enhancers, osmotic pressure maintenance agents, absorption retarders, diluents, adjuvants, preservatives, and stabilizers, etc. For example, the pH regulator includes but is not limited to phosphate buffer solutions. The surfactant includes but is not limited to cationic, anionic, or non-ionic surfactants, such as Tween-80. The ionic strength enhancer includes but is not limited to sodium chloride. The osmotic pressure maintenance agent includes but is not limited to sugars, NaCl, and their analogues. The absorption retarder includes but is not limited to monostearate and gelatin. The diluent includes but is not limited to water, aqueous buffer solutions (e.g. buffered saline), alcohols, and polyols (e.g. glycerin), etc. The adjuvant includes but is not limited to aluminum adjuvants (e.g. aluminum hydroxide), Freund's adjuvants (e.g. complete Freund's adjuvants), etc. The preservative includes but is not limited to various antibacterial and antifungal agents, such as thiomersal, 2-phenoxyethanol, parabens, tributan, phenol, and sorbic acid, etc. The stabilizer has the meanings commonly understood by those skilled in the art, which can stabilize the expected activity of active ingredients in drugs (e.g. inhibitory activity against PSD-95 ubiquitination), including but not limited to sodium glutamate, gelatin, SPGA, sugars (e.g. sorbitol, mannitol, starch, sucrose, lactose, glucan, or glucose), amino acids (e.g. glutamic acid, or glycine), proteins (e.g. dry whey, albumin, or casein), or their degradation products (e.g. lactalbumin hydrolysates), etc.

As used herein, the term “treatment” refers to treating or curing a disease (e.g. osteoporosis), delaying the onset of one or more symptoms of the disease, and/or delaying the development of the disease.

As used herein, the term “effective amount” refers to the amount that can effectively achieve the intended purposes. For example, a therapeutically effective amount can be an amount that is effective or sufficient to treat or cure a disease (e.g. osteoporosis), delay the onset of one or more symptoms of the disease, and/or delay the development of the disease. Such an effective amount can be easily determined by those skilled in the art or physicians, and can be associated to the expected purposes, general health, age, gender, weight of the subjects, severity of the disease to be treated, complications, and administration modes, etc. The determination of such an effective amount is entirely within the capability of those skilled in the art.

As used herein, the biological function of the polypeptide or the variant thereof of the present invention includes but is not limited to promoting bone formation, bone regeneration, bone repair, and/or preventing and/or treating osteoporosis.

### Beneficial Effects

1) The WNT7B-derived polypeptides designed by the present invention can significantly promote osteogenic differentiation of human mesenchymal stem cells in vitro, and have more significant effects compared to the existing recombinant WNT3A proteins in clinical practice, without activating the classical WNT pathway. In view of this, it is expected to address the serious side effects (tumorigenesis, etc.) that may be caused by the in vivo use of the existing WNT agonists, laying foundation for the implementation of the subsequent in vivo experiments;

(2) The WNT7B-derived polypeptides designed by the present invention can significantly improve two types of osteoporosis through in vivo injection, including estrogen related osteoporosis (ovariectomy model) and age-related osteoporosis (age-related aging model) in mice;

(3) The application of WNT7B-derived polypeptides in the critical bone defect model of large mammals-miniature pigs has achieved ideal bone regeneration outcomes. Through sequencing and other molecular biology experiments, it is confirmed that during this process, multiple MSC subpopulation are mobilized to invade and conduct osteogenic differentiation, during which the classical WNT signaling pathway is not activated, indicating that the WNT7B-derived polypeptides have cross-species therapeutic potentials;

(4) WNT7B-derived polypeptides do not activate the classical WNT signaling pathway both in vivo and in vitro, but enhance the self-renewal and osteogenic differentiation of MSCs through the activation of Reck/Gpr124 signalsome mediated Ca2+/Nfatc1.

The embodiments of the present invention will be described in detail below in conjunction with the accompanying drawings and examples. However, those skilled in the art will understand that the following accompanying drawings and examples are only used to illustrate the present invention and not to limit the scope of the present invention. The various objectives and advantageous aspects of the present invention will become apparent to those skilled in the art based on the following detailed descriptions of the accompanying drawings and preferred embodiments.

### BRIEF DESCRIPTION OF THE DRAWINGS

FIG. 1 shows the development of WNT7B-derived polypeptides. FIG. 1 panel A shows the predicted structural picture of WNT7B protein. Wherein, the positions of Thumb and index domains are specifically marked with dashed lines; FIG. 1 panel B shows the sequences of single Thumb and index domains of human WNT7B protein; and FIG. 1 panel C shows the sequences of the derived peptide 1797 and its variants 1798, 1799, and 1800 designed in accordance with the present invention.

FIG. 2 shows the in vitro bone formation promoting effect of the WNT7B-derived polypeptides. FIG. 2 panel A shows the quantitative results of Alizarin Red staining of human mesenchymal stem cells (hMSCs) treated with four derived polypeptides at different concentrations (cultured in osteogenic induction medium (OM) for 28 days); and FIG. 2 panel B shows the evaluation of key osteogenic gene levels by RT-qPCR.

FIG. 3 shows further validation of the bone formation promoting effect of 1797 polypeptide in in vitro simulated bone engineering system. FIG. 3 panel A demonstrates the mineralized nodules of hMSCs after inducing with OM medium on a 1797 scaffold for 21 days; and FIG. 3 panel B demonstrates the distribution of C, O, Ca, and P elements after 21 days.

FIG. 4 shows significant remission of osteoporosis by in vivo injection of 1797 polypeptide in two models. FIG. 4 panel A shows the μCT images of lumbar vertebra in an ovariectomy-mediated estrogen-related osteoporosis model of mice after different treatments; and FIG. 4 panel B shows the representative μCT images of the trabecular bone of femur in the age-related osteoporosis model.

FIG. 5 shows the single cell sequencing results after applying 1797 polypeptide scaffold in the critical bone defect model of miniature pigs. FIG. 5 panel A demonstrates the differentiated cell population that appear after dimensionality reduction analysis of mesenchymal stem cell groups;

FIG. 5 panel B demonstrates the number of cells of MSC subpopulation in the control group and 1797 group; and FIG. 5 panel C shows gene set enrichment analysis (GSEA) data for β-Catenin-dependent classical WNT signaling within the MSC lineage in the control group and 1797 group.

FIG. 6 shows immunofluorescent staining of samples obtained from the critical bone defect model of mice (activated β-Catenin, ABC). FIG. 6 panel A shows the statistical results of the number of positive cells per unit area; FIG. 6 panel B shows the activation level of the WNT signaling pathway detected by TOP/FOP dual-luciferase assay; and FIG. 6 panel C demonstrates the level of β-Catenin dependent characteristic genes in hMSCs analyzed by RT-qPCR. FIG. 6 panel D shows the violin plot of Nfatc1 expression in the MSC lineage cell group in the critical bone defect model of miniature pigs; and FIG. 6 panel E summarizes the related mechanisms by which the 1797 polypeptides play the bone formation promotion role without activating the classical WNT pathway.

FIG. 7 shows evaluation of bone formation promoting effect after applying 1797 polypeptide in the age-related osteoporosis model of miniature pigs in Example 7.

FIG. 8 shows analysis of bone formation promoting effect after applying 1797 polypeptide scaffold in the critical bone defect model of miniature pigs in Example 8. FIG. 8 panel A demonstrates the effect after transplanting 1797 polypeptide scaffold; FIG. 8 panel B shows the relative mineralization degree of the defect area 6 months after surgery (statistical analysis based on μCT results); FIG. 8 panel C shows the representative images of H&E staining; and FIG. 8 panel D shows the results of animal behavioral evaluation after implantation of a control scaffold and 1797 polypeptide scaffold.

### DETAILED DESCRIPTION

The present invention will be further illustrated below in conjunction with specific examples, however the examples do not limit the present invention in any way. Unless otherwise specified, the reagents, methods, and equipment used in the present invention are conventional reagents, methods, and equipment in the technical field.

### Example 1: Development of WNT7B-Derived Polypeptides

A series of polypeptides were designed based on the Thumb and index domains of WNT7B in the present invention, and the specific experimental processes are as follows:

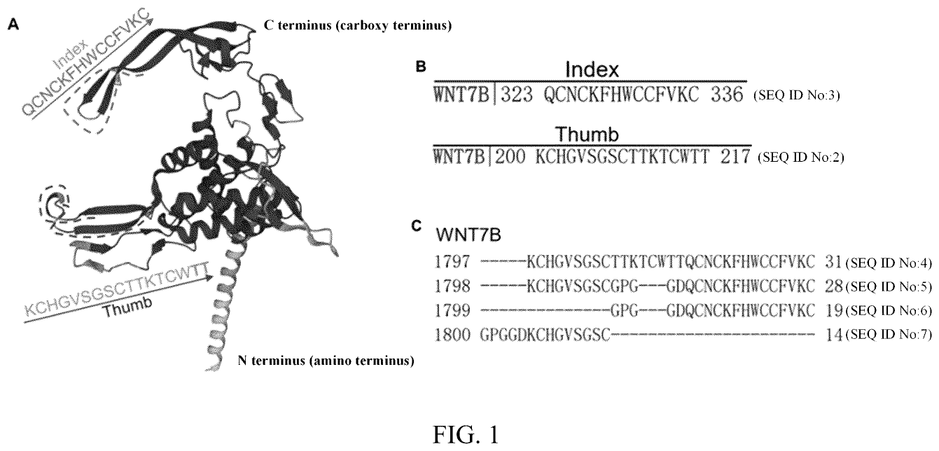

(1) Thumb and index domains were essential for the ligand selective signaling of WNT7B. The Thumb domain was located near the N-terminus (amino terminus) and had the sequence of KCHGVSGSCTTKTCWTT (SEQ ID NO:2), and the index domain was located near the C-terminus (carboxyl terminus) and had the sequence of QCNCKFHWCCFVKC (SEQ ID NO:3) (FIG. 1 panel A-panel B);

(2) The polypeptides were designed based on WNT7B Thumb and index domains. As shown in FIG. 1 panel C, the polypeptide 1 (1797) involved was composed of 31 amino acids, and the specific sequence was KCHGVSGSCTTKTCWTTQCNCKFHWCCFVKC (SEQ ID NO:4); the variant polypeptide 2 of polypeptide 1 (1798) was composed of 28 amino acids, and the sequence was KCHGVSGSCGPGGDQCNCKFHWCCFVKC (SEQ ID NO:5); variant polypeptide 3 (1799) was composed of 19 amino acids, and the sequence was GPGGDQCNCKFHWCCFVKC (SEQ ID NO:6); and variant polypeptide 4 (1800) was composed of 14 amino acids, and the sequence was GPGGDKCHGVSGSC (SEQ ID NO:7).

All polypeptides of the present invention were outsourced to the company and synthesize by chemical methods.

### Example 2: In Vitro Bone Formation Promoting Effect of the WNT7B-Derived Polypeptides

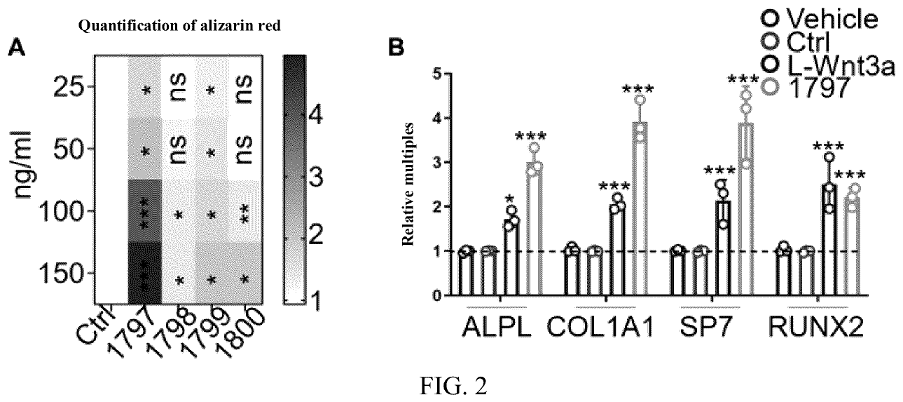

Evaluation of in vitro bone formation promoting effects was carried out using the polypeptides and their variants designed in Example 1. The results are shown in FIG. 2, indicating that this series of polypeptides have good in vitro bone formation promoting effects, among which 1797 has the best effect. The specific experimental processes are as follows:

(1) the human mesenchymal stem cells (hMSCs) were cultivated in osteogenic induction medium (OM) for 28 days;

(2) the medium was removed, the cells were washed twice with 1×PBS (containing no calcium and magnesium ions), and then the cells were fixed at room temperature for 15 minutes;

(3) the fixative solution was removed, the cells were washed twice with PBS again, then the sample was covered with Alizarin Red staining and incubated at 37° C. in the dark for 60 minutes;

(4) After the excess dye liquor was washed off with PBS, Alizarin Red was quantitatively analyzed. As shown in FIG. 2, 1797 has the best bone formation promoting effect in hMSCs cultured in vitro, which has significant difference compared to the control group. Although other variants also have certain bone formation promoting effects, their significance is lower than 1797;

(5) RNA extraction, cDNA synthesis, and PCR amplification were performed on hMSCs cultured under the same conditions as above. The levels of key osteogenic genes, including ALPL, COL1A1, RUX2, and SP7, were detected by RT-qPCR, and recombinant WNT3A protein was used as a positive control; Compared with the recombinant WNT3A protein, the 1797 polypeptide designed in the present invention has a stronger osteogenic induction effect on hMSCs.

### Example 3: Evaluation of Bone Formation Promoting Effect of 1797 Polypeptide in Human Cells

The promoting effect of 1797 polypeptide on osteogenic differentiation of hMSCs was observed in an in vitro simulated bone engineering system. The specific experimental processes are as follows:

(1) Construction of in vitro simulated bone engineering system:
a. soaking the pure hyaluronic acid (HA) scaffold in solution of graphene oxide (GO) in alcohol for 30 minutes and drying same at 60° C. to obtain the HA/GO scaffold;
b. further soaking HA/GO in a composite solution (a ratio of 1:4 of ethylenediamine (EDA) to dimethylformamide (DMF)) at 40° C. for 12 hours, and grafting the amino group —NH₂ onto the carboxyl group-COOH of GO to obtain HA/GO-NH₂;
c. soaking the HA/GO-NH₂ scaffold in a 2 wt % sodium ascorbate (NaVC) aqueous solution at a condition of 60° C. for 8 hours to reduce additional oxidative functional groups on GO-HN₂; and
d. soaking HA/rGO-NH₂ in 1797 polypeptide aqueous solution (2 mg/mL) at a condition of 15° C. for 48 hours to prepare the HA/rGO-1797 scaffold;

(2) the modified HA/rGO-1797 scaffold was put in a well plate, hMSCs were inoculated onto the scaffold, and the cells were cultivated in OM medium for 21 days;

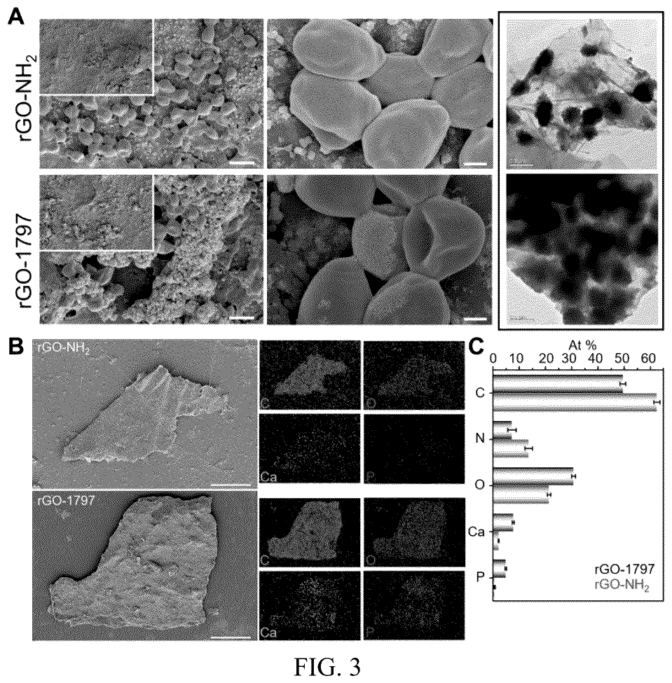

(3) After 21 days, the deposition of mineralized nodules on the 1797 scaffold and control scaffold was observed by transmission electron microscopy (TEM). The results in FIG. 3 panel A showed a significant increase in mineralized nodules on the 1797 scaffold compared to the control scaffold, indicating that 1797 promoted the mineralization of hMSCs;

(4) Element mapping showed that the content of Ca and P elements on the 1797 scaffold was significantly increased compared to the control scaffold (FIG. 3 panel B), indicating that the 1797 polypeptide can significantly enhance the bioosteogenesis and mineralization of hMSCs.

### Example 4: Evaluation of Bone Formation Promoting Effect of 1797 Polypeptide in Osteoporosis Model of Mice

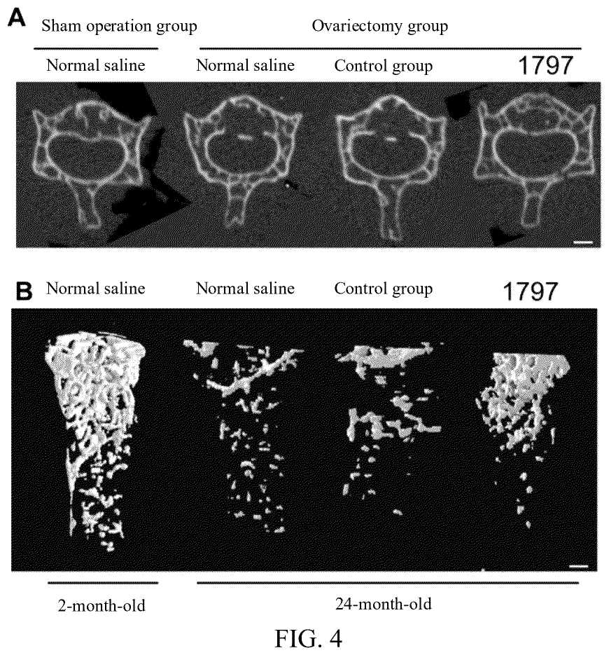

As shown in FIG. 4, the 1797 polypeptide designed by the present invention plays the bone formation promoting effect in both osteoporosis models of mice, and efficiently alleviating the progression of diseases. The specific experimental processes are as follows:

(1) Establishment of ovariectomy (OVX)-mediated osteoporosis model:
a. After intraperitoneal anesthesia with 3% pentobarbital sodium, lying the mice in a prone position, with the highest point of the mouse's back vertebral column as the starting point, and dehairing on the back at a distance of about 1 cm from left and right;
b. under sterile conditions, bluntly separating the skin and muscles in the depilation area, and observing white adipose tissue through the parietal peritoneum to locate the ovarian site of mouse;
c. using 1-gauge suture to perform encircling ligation at the junction of ovaries and fallopian tubes, and using surgical scissors to remove the ovaries;
d. pushing the adipose tissue back into the abdominal cavity, suturing the tectorial membrane and muscular layer together, and then suturing the skin;
e. subjecting the Sham operation group to identical procedures as the experimental group (excluding those receiving ovariectomy);
f. euthanizing the experimental mice two months after surgery and proceeding with further analysis.

(2) Establishment of age-related osteoporosis model: the mice survived in SPF environment for 24 months, during which they were placed in a well-equipped facility at a temperature of 20-25° C. and humidity of 30%-70%, under the same dark/light cycle (12:12). After 24 months, the mice were euthanized and the next experimental procedure was proceeded.

(3) The results in FIG. 4 showed that the in vivo injection of 1797 polypeptide significantly alleviated estrogen deficiency-related (OVX model) and age-related osteoporosis in mice, specifically manifested as: as can be seen from μCT images, compared with the control group, the bone trabecular mass of mice in the 1797 treatment group increased, ultimately eliminating the osteoporosis phenotype of the OVX group (FIG. 4 panel A);

(4) In the age-related osteoporosis model, injection of 1797 polypeptide significantly increased the bone trabecular mass in aged mice and effectively alleviated osteoporosis (FIG. 4 panel B).

### Example 5: Evaluation of Effect of 1797 Polypeptide Scaffold in the Critical Bone Defect Model of Miniature Pigs

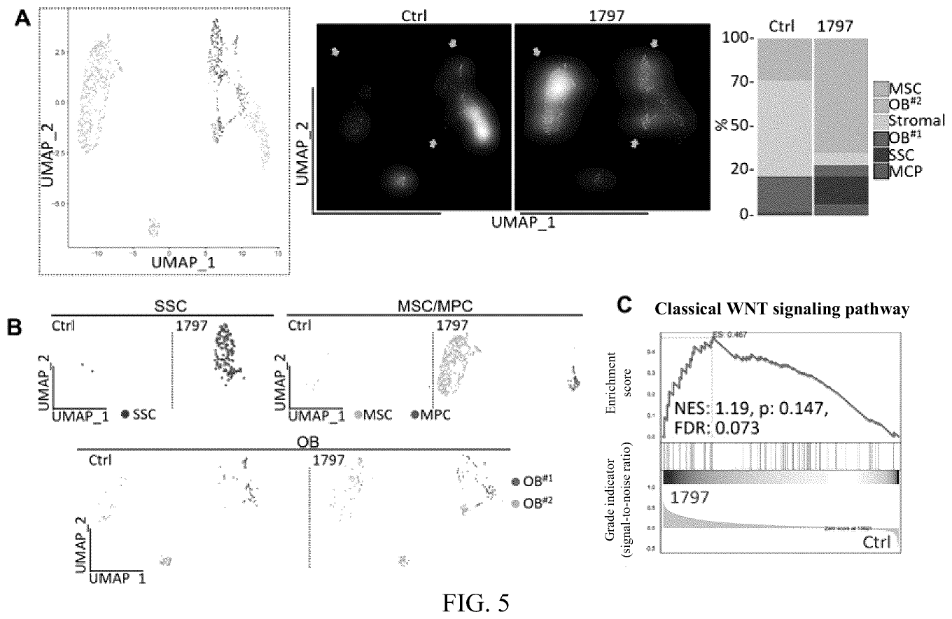

The 1797 polypeptide scaffold was applied the critical bone defect model of large mammals, and the mechanism of promoting bone formation and bone repair was verified by single-cell sequencing, which was the response of the MSCs population. The specific experimental processes are as follows:
(1) Construction of critical bone defect model using miniature pigs;
(2) the engineered 1797 scaffold was transplanted into the bone defect cavity, the scaffold was acquired and digested one month after transplantation, and single-cell RNA sequencing (scRNA-seq) was performed on the digested cells;
(3) From FIG. 5, it can be seen that in the mesenchymal components of the 1797 polypeptide scaffold treatment group, the cell subpopulation that showed significant differences compared to the control group were skeletal stem cells (SSCs), mesenchymal stem cells (MSCs), and mesenchymal progenitor cells (MPCs). There were almost no MSC, SSC, and MPC subgroups in the control group, but the number of these subgroups significantly increased in 1797 group (FIG. 5 panel A);
(4) The subpopulation of osteoblasts (OB) in the 1797 polypeptide scaffold group also increased, showing enhanced bone formation (FIG. 5 panel B);
(5) Gene set enrichment analysis (GSEA) was performed on the mesenchymal fraction, and compared with the control group, the 1797 polypeptide scaffold group did not exhibit typical WNT activation, indicating that the in vivo effect of 1797 on MSCs was β-Catenin independent (FIG. 5 panel C).

### Example 6: Non-Activation of Classical WNT Signaling Pathway by 1797 Polypeptide

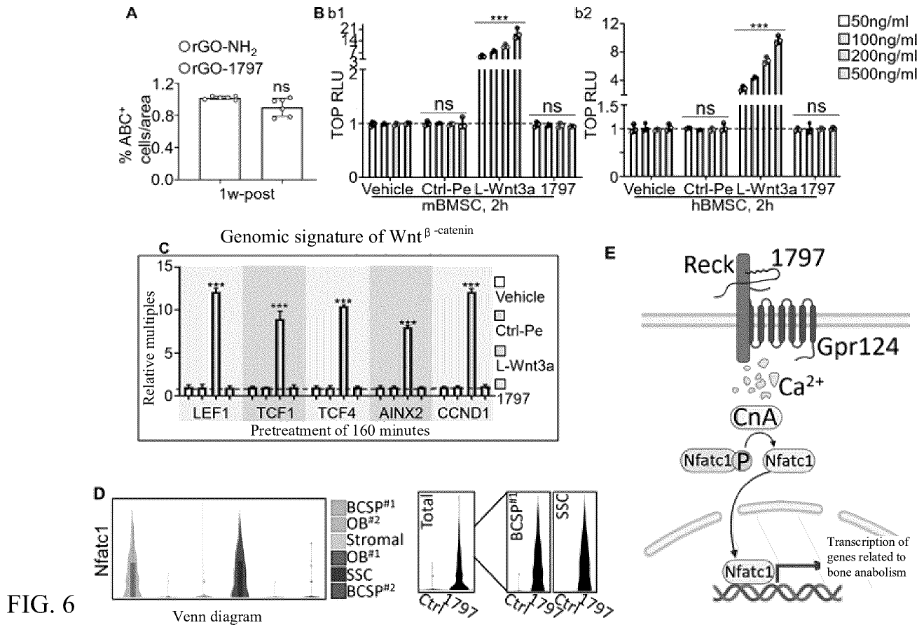

It was further confirmed that 1797 polypeptide didn't activate the classical WNT signaling pathway both in vivo and in vitro, but enhanced the self-renewal and osteogenic differentiation of MSCs through the activation of Reck/Gpr124 signalsome mediated Ca2+/Nfatc1. The specific experimental processes are as follows:
(1) Construction of critical bone defect model of mice;
(2) Decalcification of samples was carried out with 10%-12% EDTA. After the samples were softened, they were dehydrated overnight with 30% sucrose solution at 4° C.;
(3) the samples were presoaked with OCT at 4° C. for 3 hours, the angle of the samples was adjusted, which were then frozen and embedded at −20° C. After the samples were frozen, they were sliced with freezing microtome, with a thickness of 6 μm;
(4) Immunofluorescent staining: Frozen sections of tissues were soaked in PBS for 15 minutes. At room temperature, the membrane was permeated by 0.5% Triton for 10 minutes. BSA powder was dissolved in 0.5% Triton to obtain a Triton solution containing 3% BSA for sample blocking. The primary antibody (activated β-Catenin, ABC) was diluted according to the concentration recommended in the instructions, and incubated overnight at 4° C. The primary antibody was washed with PBS, the secondary antibody and the DAPI solution were diluted according to the concentration recommended in the instructions, and incubated at room temperature for 2 hours. The secondary antibody was washed with PBS for three times, 10 minutes per time. Finally, antifade solution was used to seal the film, images were obtained through laser confocal microscopy, and statistical analysis was performed by ImageJ;
(5) The number of cells expressing ABC per unit area was counted (FIG. 6 panel A), and the results showed that the 1797 polypeptide did not activate β-Catenin in mice;
(6) The activation of the WNT pathway was detected by TOP/FOP dual-luciferase assay. FIG. 6 panel B shows that compared with recombinant WNT3A, the treatment with 1797 polypeptide will not activate the classical WNT signaling pathway in both human- and mouse-derived mesenchymal cell lines;
(7) It was further verified through RT-qPCR that the treatment with 1797 did not induce β-Catenin dependent gene expression in hMSCs compared to recombinant WNT3A (FIG. 6 panel C);
(8) The results of single-cell sequencing of cells grown after implantation of 1797 polypeptide scaffold in the critical bone defect model of miniature pigs in Example 5 were further analyzed, and it was found that the Nfatc1 positive cell population were significantly up-regulated in the MSC lineage (FIG. 6 panel D);
(9) Previously, it has been reported that the self-renewal and osteogenic differentiation of Nfatc1+MSC are controlled by Ca2+/Nfatc1 signaling. Therefore, the 1797 polypeptide can induce the self-renewal and osteogenic differentiation of Nfatc1+MSC through the activation of Reck/Gpr124 signalsome mediated Ca2+/Nfatc1, thereby exerting in vitro and in vivo bone formation promoting effects (FIG. 6 panel E).

### Example 7: Evaluation of Bone Promoting Effect of 1797 Polypeptide in Age-Related Osteoporosis Model of Miniature Pigs

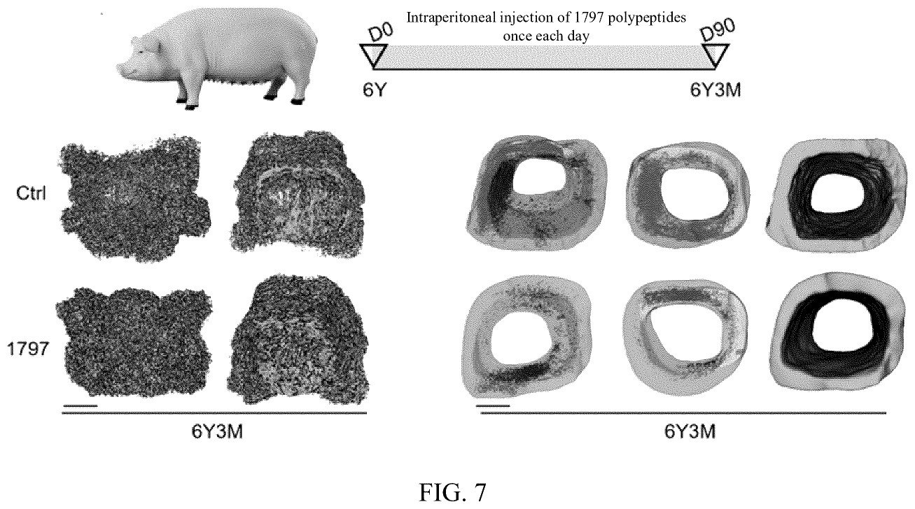

This example analyzed the alleviation and bone promoting effects of WNT7B-derived polypeptide 1797 on disease progression in age-related osteoporosis model of miniature pigs. The specific experimental processes are as follows:
(1) Throughout the experiment, the miniature pigs were placed under a constant light/dark schedule of 12 hours/12 hours, and a constant temperature of 20° C. and a constant relative humidity of 50% were maintained;
(2) Miniature pigs can drink water freely in addition to receiving granular commercial food twice a day;
(3) Animal suffering should be minimized and the number of animals used should be reduced during the experimental process;
(4) All animals were monitored by veterinarians during the experiment;
(5) 1797 polypeptide was intraperitoneally injected daily for three consecutive months since 6-year-old of miniature pigs. After three months, the animals were euthanized and femoral tissue was obtained for CT analysis and reconstruction;
(5) The μCT images in FIG. 7 showed that compared with the control group, the in vivo application of 1797 polypeptide significantly increased the number, thickness, and bone mass of bone trabeculae in aged pigs, while reducing the separation of bone trabeculae in the distal femoral metaphysis; and 1797 continuously reduced the porosity of cortical bone in aged pigs and increased the bone mineral density of cortical bone.

This example demonstrates that applying WNT7B-derived polypeptide 1797 to age-related osteoporosis model of miniature pigs can effectively alleviate disease symptoms and increase bone mass.

### Example 8: Effects on Bone Regeneration and Animal Recovery by the Application of 1797 Polypeptide in the Critical Bone Defect Model of Miniature Pigs

In this example, the WNT7B-derived polypeptide 1797 was applied in the critical bone defect model of miniature pigs to observe the effects of bone regeneration and animal recovery. The specific experimental processes are as follows:

(1) Construction of critical femoral fracture model using miniature pigs:
a. To ensure adaptation to the environment, isolating the miniature pigs for five to ten days before the experiment;
b. According to ethical standards, euthanizing the miniature pigs within 6 months after scaffold implantation. Separating the femoral condyle and using a scalpel and scissors to cut open the skin and muscles. Using a circular saw to remove the intact condyle of the femur;
c. Fixing the tissue specimens in 4% neutral buffered formalin at room temperature on a 100 mL shake table for five days;
d. During the surgery, giving general anesthesia and intravenously administering prophylactic amoxicillin (20 mg/kg; Centrafarm). Preoperative injection of meloxicam (15 mg/mL, Metacam; Boehringer) as an analgesic; in addition, to reduce the risk of infection, antibiotics (streptomycin 0.5 g/day) should be taken one hour before anesthesia induction and within two days after anesthesia induction;
e. Wiping the surgical site with 4% chlorhexidine gluconate surgical scrub for surface disinfection, and then making a longitudinal incision on the inner skin of the right knee;
f. Cutting open the subcutaneous tissue and periosteum, exposing the surface of the femoral condyle bone, and using a bone trephine to prepare a cavity at a speed of 2000 revolutions per minute under saline flushing (0.9% NaCl). The exposed bone cavity diameter of each animal was 10 mm and the depth was 6 mm;

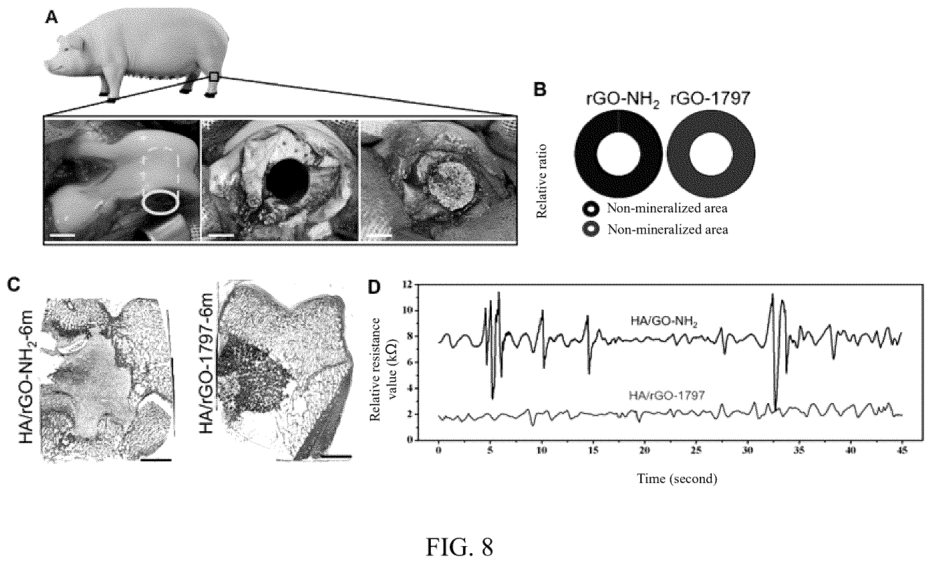

(2) transplanting the 1797 polypeptide scaffold into the above-mentioned femoral cavity, and performing micro-CT analysis 6 months after transplantation. The μCT data in FIG. 8 showed that there was a large amount of new bone in the 1797 polypeptide scaffold group, while there was no significant regenerated bone tissue in the control group; the histological staining results showed that 6 months after transplantation, the control group exhibited significant rejection reactions, filled with non-mineralized fibrous tissue, while the defect area of the 1797 polypeptide scaffold group had been occupied by new bone, and the scaffold had integrated with the host bone as a whole, a large amount of new bone was also regenerated in the scaffold pores, indicating that the stem cells expanded and differentiated towards bone formation in the microenvironment where 1797 polypeptide was present. The functional activity testing showed that 1797 polypeptide scaffold treatment restored almost the normal activity in miniature pigs with bone defects, while the control group still had poor activity. This example demonstrates that the application of 1797 polypeptide in the critical bone defect model of miniature pigs can effectively promote new bone formation, restore normal activity ability of animals, and have good biocompatibility.

It should be noted that the preferred embodiments of the present invention are provided in the specification, however the present invention can be implemented in many different forms and is not limited to the embodiments described in the specification. These embodiments are not intended to be additional limitations on the content of the present invention, but are provided for the purpose of providing more thorough and comprehensive understandings of the disclosure of the present invention. Moreover, the above-mentioned technical features are continued to be combined with each other to form various embodiments not listed above, all of which are considered to be within the scope recorded in the specification of the present invention. Further, for those of ordinary skill in the art, improvements or transformations can be made based on the above descriptions, and all such improvements and transformations should fall within the scope of protection of the attached claims in the present invention.

1. An isolated polypeptide or a variant thereof, wherein the polypeptide consists of amino acids at positions 200-217 and amino acids at positions 323-336 of WNT7B protein,
wherein the variant differs from the polypeptide from which the variant is derived in substitutions, deletions, and/or additions of one or more amino acid residues, and retains biological function of the polypeptide from which the variant is derived.

2. The isolated polypeptide or the variant thereof according to claim 1, wherein the WNT7B protein has an amino acid sequence as shown in SEQ ID NO:1, and the isolated polypeptide comprises an amino acid sequence as shown in SEQ ID NO:4.

3. The isolated polypeptide or the variant thereof according to claim 1, wherein the variant comprises an amino acid sequence as shown in SEQ ID NOs: 5-7.

4. A fusion protein, comprising the isolated polypeptide or the variant thereof according to claim 1 and an additional polypeptide;
in particular, the additional polypeptide is linked to the N-terminus or C-terminus of the isolated polypeptide or the variant thereof optionally via a linker; and
in particular, the additional polypeptide is selected from a protein tag, a targeting moiety, or any combinations thereof.

5. An isolated nucleic acid molecule, comprising a nucleotide sequence encoding the isolated polypeptide or the variant thereof according to claim 1.

6. A vector, comprising the isolated nucleic acid molecule according to claim 5.

7. A host cell, comprising the isolated nucleic acid molecule according to claim 5.

8. A pharmaceutical composition, comprising the isolated polypeptide or the variant thereof according to claim 1, and a pharmaceutically acceptable carrier and/or excipient.

9. The pharmaceutical composition according to claim 8, wherein the pharmaceutical composition optionally further comprises an additional pharmaceutically active agent; and
in particular, the additional pharmaceutically active agent is an other drug that has effects of promoting bone formation, bone regeneration, and/or bone repair.

10. Use of the isolated polypeptide or the variant thereof according to claim 1 in preparation of a drug, wherein the drug is configured to promote bone formation, bone regeneration, and bone repair, and/or prevent and/or treat osteoporosis.
